(19) United States
(12) Patent Application Publication
(10) Pub. No.: US 2021/0390840 A1
(43) Pub. Date: Dec. 16, 2021

(54) SELF-SUPERVISED SOCIAL DISTANCE DETECTOR

(71) Applicant: 3D Industries Limited, London (GB)

(72) Inventors: Seena Hossein REJAL, London (GB); Sukrit SHANKAR, London (GB); Raj Neel SHAH, London (GB)

(21) Appl. No.: 16/899,550

(22) Filed: Jun. 11, 2020

(51) Int. Cl. G08B 21/18; G01B 11/14; G06K 9/62; H04W 4/02; G06K 9/00; G06N 3/08; G06N 3/04; B64C 39/02; B64D 47/08

(52) U.S. Cl. CPC G08B 21/182; G01B 11/14; G06K 9/6259; H04W 4/023; G06K 9/00711; G06K 9/00335; G16H 50/80; G06K 9/00288; G06K 9/6262; G06N 3/088; G06N 3/04; B64C 39/024; B64D 47/08; G06K 9/0063

(57) ABSTRACT

In various examples there is an apparatus with a memory storing a video captured by a capture device, the video depicting a scene comprising two or more people in an environment. The apparatus has a self-supervised neural network which takes at least one frame of the video as input and in response, computes a prediction of at least four points in the frame which depict four points on a plane of the scene. A processor computes, from the four points, a plan view of the scene and detects two or more people in the plan view of the scene. The processor computes, for individual pairs of people depicted in the plan view, an estimate of shortest distance between the people in the pair.

### BACKGROUND

There is an increased need to maintain social distancing whereby people reduce social contact in order to prevent spread of disease. Authorities may specify social distancing guidelines and rules for people to adhere to such as specifying how many people are allowed to be present in a group, a minimum distance apart for people to be, and requirements concerning wearing face masks in particular environments.

It is often difficult for individuals to adhere to social distancing requirements for a variety of reasons, including forgetfulness, lack of understanding in the case of individuals with dementia or learning disabilities, and other reasons such as being distracted.

It is difficult for those managing public health to understand when and where social distancing is being applied and to plan and manage public health services.

The embodiments described below are not limited to implementations which solve any or all of the disadvantages of known social distance detectors.

### SUMMARY

The following presents a simplified summary of the disclosure in order to provide a basic understanding to the reader. This summary is not intended to identify key features or essential features of the claimed subject matter nor is it intended to be used to limit the scope of the claimed subject matter. Its sole purpose is to present a selection of concepts disclosed herein in a simplified form as a prelude to the more detailed description that is presented later.

In various examples there is an apparatus with a memory storing a video captured by a capture device, the video depicting a scene comprising two or more people in an environment. The apparatus has a self-supervised neural network which takes at least one frame of the video as input and in response, computes a prediction of at least four points in the frame which depict four points on a plane of the scene. A processor computes, from the four points, a plan view of the scene and detects two or more people in the plan view of the scene. The processor computes, for individual pairs of people depicted in the plan view, an estimate of shortest distance between the people in the pair.

Many of the attendant features will be more readily appreciated as the same becomes better understood by reference to the following detailed description considered in connection with the accompanying drawings.

### DESCRIPTION OF THE DRAWINGS

The present description will be better understood from the following detailed description read in light of the accompanying drawings, wherein:

FIG. 1 is a schematic diagram of an image/video analysis apparatus for social distancing applications;

FIG. 2 is a schematic diagram of an information dashboard at a control centre;

FIG. 3 is a flow diagram of a method of operation of an image/video analysis apparatus for social distancing applications;

FIG. 4 is a flow diagram of a method of creating training data and a method of using the training data to train a classifier;

FIG. 5 is a flow diagram of a method of depth estimation;

FIG. 6 is a flow diagram of a method of computing absolute distances;

FIG. 7 is a flow diagram of a method of self-supervised training of a neural network;

FIG. 8 illustrates an exemplary computing-based device in which embodiments of an image/video analysis apparatus for social distancing applications are implemented.

Like reference numerals are used to designate like parts in the accompanying drawings.

### DETAILED DESCRIPTION

The detailed description provided below in connection with the appended drawings is intended as a description of the present examples and is not intended to represent the only forms in which the present example are constructed or utilized. The description sets forth the functions of the example and the sequence of operations for constructing and operating the example. However, the same or equivalent functions and sequences may be accomplished by different examples.

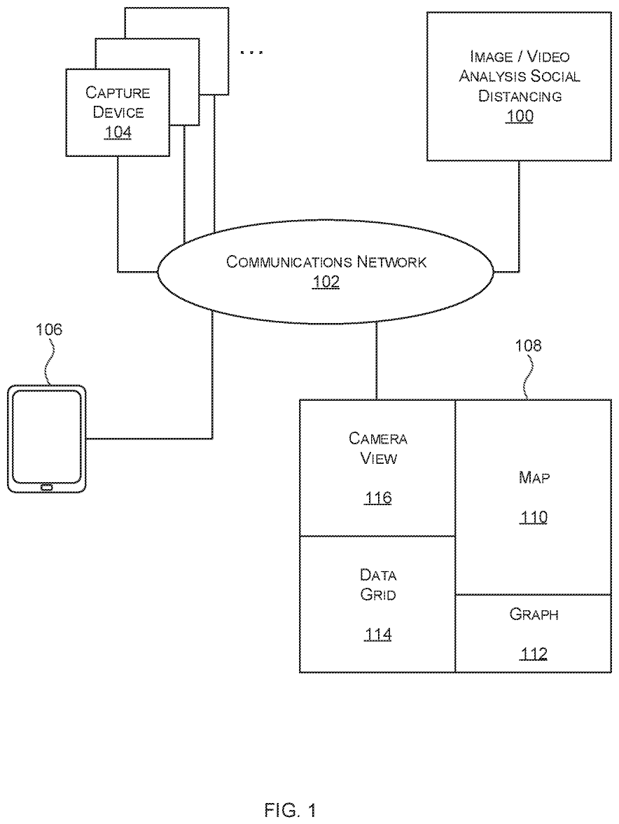

FIG. 1 is a schematic diagram of an image/video analysis apparatus 100 deployed as a cloud service. The image/video analysis apparatus 100 is in communication with one or more capture devices 104, 106 via communications network 102. A non-exhaustive list of examples of a capture device 104 is: drone camera, closed circuit television camera, surveillance camera, web camera, smart phone camera, video camera. It is possible to use combinations of different types of capture device 104. Images and/or video streams are sent from the capture devices 104, 106 to the image/video analysis apparatus 100 over communications network 102 using encryption and data compression where appropriate.

In some cases the capture devices comprise pre-existing networks of video surveillance cameras deployed at geographical locations in towns and cities through a region or state. In some cases the capture devices are deployed specifically for use with the image/video analysis apparatus 100 of FIG. 1. Hybrids of these approaches are possible.

The capture devices may be deployed at a variety of different types of location. A non-exhaustive list of examples of location is: hospital, school, station, port, airport, enterprise premises, construction site, public park.

The image/video analysis apparatus 100 processes the received images and/or videos to detect breaches of social distancing guidelines and/or to collect data concerning social distancing behaviors. In some embodiments the image/video analysis apparatus 100 outputs a user interface 108 displayable at any computing device in communication with the apparatus 100 over the communications network 102. The user interface 108 comprises a display 116 of current output of a capture device, a map 110 showing frequency of occurrence of social distancing breaches by location, a graph 112 of social distancing breaches by day, and a data grid 114 of social distancing data computed by the apparatus 100 for image/video data obtained from capture devices 104 in different geographical locations.

The image/video analysis apparatus 100 facilitates detection of when and where people are not social distancing. In some examples it enables tracing of initial cases spread by people without face masks. It is able to facilitate identification of geographical locations predicted to need more medical staff and tests for the disease. In some cases the image/video analysis apparatus 100 is arranged to generate reports for compliance purposes.

In some examples the image/video analysis apparatus 100 detects a direction of movement of people in an environment and triggers a social distancing breach alert if an individual is detected moving in a direction different from a specified direction. This enables “one-way direction” compliance to be assessed.

In some examples the image/video analysis apparatus 100 computes a social distancing score or index. The score or index is computed from one or more factors such as a rate of detected breaches of social distancing guidelines, an average number of detected breaches of social distancing guidelines, a frequency of detected breaches of social distancing guidelines. The score or index is sent from the image/video analysis apparatus 100 via communications network 102 to one or more end user devices. In an example it is displayed as a live score for a given facility or premises on a display that is easy for individuals to observe. In this way individuals are able to tell whether it is a good time for them to enter the facility or premises. In an example the score or index is incorporated in metadata associated with locations on an online map such that end users who view the map are able to access the metadata and find out the live score of locations and premises and/or find historic scores of locations and premises. In an example, an end of day average score is given via the online map for individual locations and/or premises. In some cases the score takes into account inputs from thermal cameras detecting body temperature.

In some examples, the image/video analysis apparatus 100 triggers an alert in response to detecting breach of a social distancing guideline. The alert is a push notification pushed to a mobile computing device such as a smart phone 106 in some cases.

In some examples the image/video analysis apparatus 100 is in communication with a web server deploying a contact tracing application. Information from the image/video analysis apparatus 100 is made available to the contact tracing application.

The image/video analysis apparatus 100 of the disclosure uses neural networks and computes a plan view of a scene from which distances between people are computed in an unconventional manner to achieve detecting of social distance guideline breaches. The plan view of the scene is also referred to as a “bird's eye view”.

FIG. 1 shows the image/video analysis apparatus 100 deployed as a cloud service. It is possible to modify the arrangement of FIG. 1 to move some or all of the functionality, of the image/video analysis apparatus 100 to a client device such as smart phone 106 or camera. In some cases the image/video analysis apparatus 100 is deployed as part of an operating system of a computing device.

Alternatively, or in addition, the functionality described herein is performed, at least in part, by one or more hardware logic components. For example, and without limitation, illustrative types of hardware logic components that are optionally used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), Graphics Processing Units (GPUs).

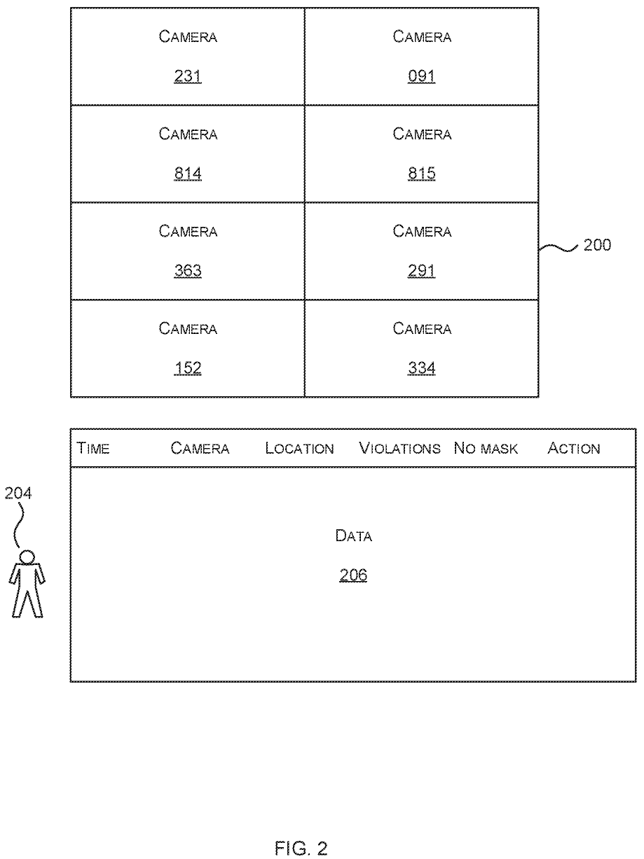

FIG. 2 is a schematic diagram of an information dashboard at a control centre staffed by operator 204. The information dashboard includes eight displays 200 of video streams. Superimposed on the video streams are marks identifying detected social distancing breaches. A separate display 206 is a table of data with analysis results computed by the apparatus 100 about the video streams. In some examples the analysis results include information about one or more of:

the existence of queues at stores and their waiting times;

assessment of occupancy levels;

effective handwashing or use of hand sanitizers;

donning of gloves;

use of appropriate personal protective equipment in hospital or other settings.

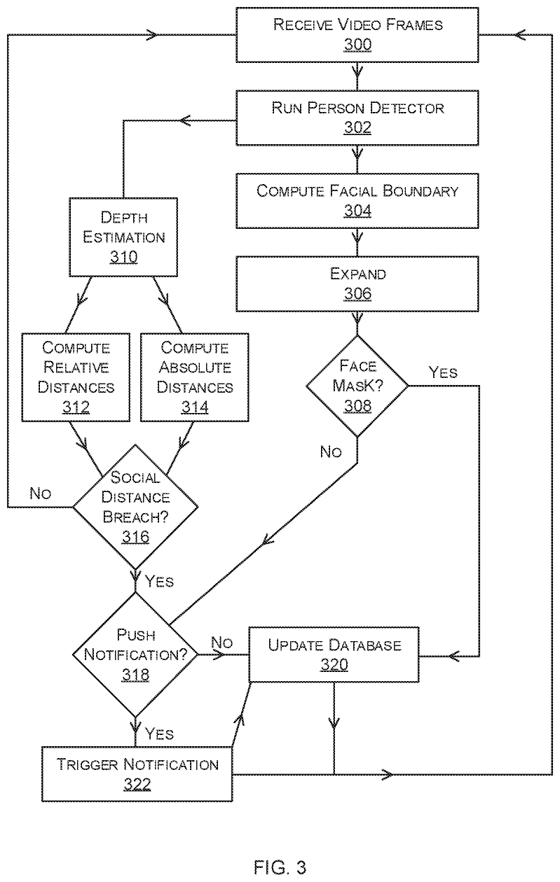

FIG. 3 is a flow diagram of a method of operation at the image/video analysis apparatus 100 of FIG. 1.

One or more video frames are received 300 from a specified capture device such as one of the capture devices 104, 106 of FIG. 1. Each video frame depicts a scene comprising an environment and optionally one or more people.

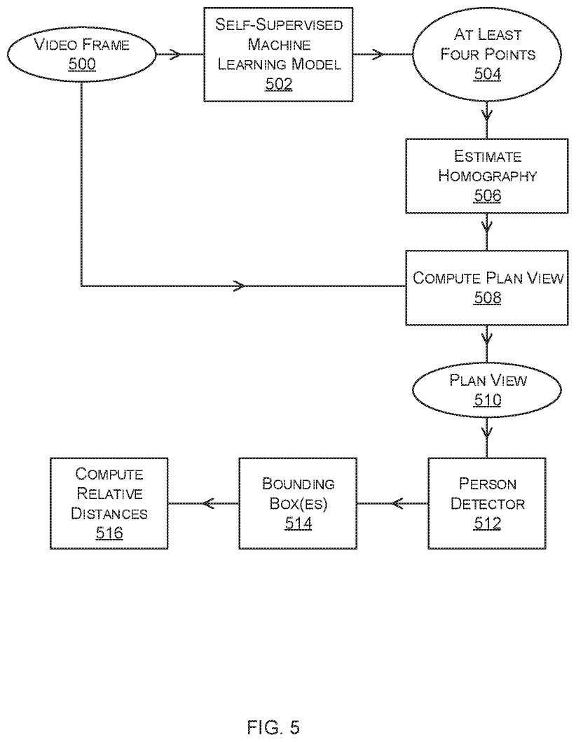

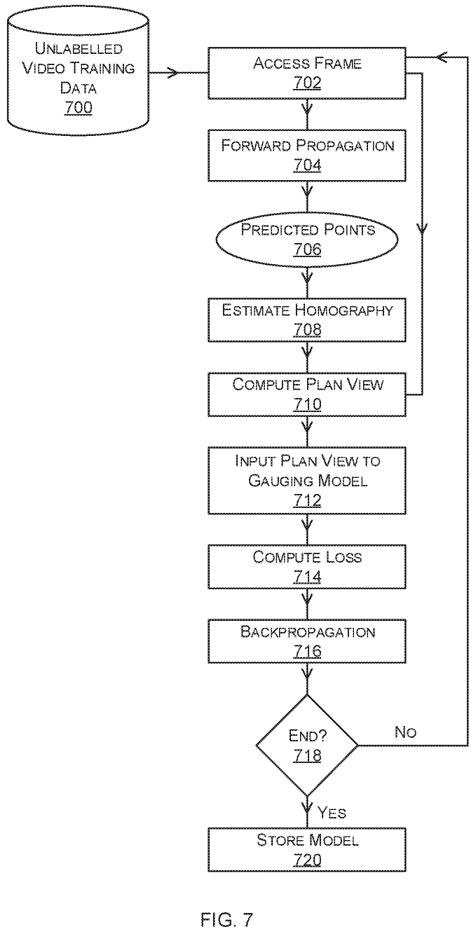

A depth estimation process 310 is then carried out. The depth estimation process is described in more detail with reference to FIG. 5 and FIG. 7 below. FIG. 5 explains how a self-supervised machine learning model 502 is used as part of a depth estimation process and FIG. 7 explains the self-supervised training of the self-supervised machine learning model 502.

To enable the technology to scale to a wide variety of videos, typical depth estimation techniques, that consist of training a (convolutional) neural network with a huge dataset annotated with depth maps are not used. Instead an approach that blends the homography estimation with deep learning is used as described with reference to FIG. 5 below.

Neural depth estimation, comprising training a (convolutional) neural network with a huge dataset annotated with depth maps, is mostly specific to the dataset it is trained upon. To collect any new training data, one has to also collect the depth maps, which may be either done through Kinect (trade mark) like sensors, or manual annotation. Both of them are not easy to be incorporated for a wide variety of incidents and situations that happen on streets. Also, for such algorithms, there is no guarantee that deep learning may perform acceptably well. A method that comes with some provable guarantees on its performance, and scales almost to all cases is preferred.

In the field of homography estimation, it is well-known that any two planes (2D rectangles) viewing a 3D scene are related by a homography. Using this concept, produce a bird's eye view (top-level view) of a given frame, which will clearly show who is behind whom and by what relative distance, and give a relative depth estimation of the scene, as one would ideally expect to get from a (perfect) neural depth estimation procedure. The approach is not at all straightforward, since for estimating the bird's eye view, one needs to locate a planar region on the 2D image of the scene, for which four planar points are at least required in the scene. Manual identification of the four planar points is time consuming and error prone.

The present technology addresses these problems by using a self-supervised approach. The concept of a self-supervised approach is to choose some attributes in the input data, and to gauge the output of that choice against some known standard, thereby learning some robust features in the process. In this way labelled training data on the original problem at hand is not essential. Detail about the self-supervised approach is given below with reference to FIGS. 5 and 7. In summary, from a given scene (operating on a frame-to-frame basis, so each scene is a red green blue (RGB) image), the process selects 4 points, which form a plane in the scene. Using the four points the process computes an estimate of a homography to a bird's eye view, and then uses the homography to transform the scene to the bird's eye view, such that some standard X is always satisfied. The standard X is the maximal probability of detection of persons from the top-view (done by training a ResNet (trade mark) on VisDrone (trade mark) dataset). Thus, the process does not need any annotation of the 4 points to use to compute the homography, or even any data for what the homography might be to a bird's eye view, given the scene structure and content. The process uses a model for detecting people in a top-view in the scene, and using this model as a gauging model, the process chooses the 4 co-ordinates on the scene, for computing the homography. The choosing of the four good planar points for homography estimation using self-supervision, is done using a ResNet (trade mark), the problem being cast as a regression problem. In this way automation of the homography estimation using a self-supervised approach with no training data is achieved. An assumption here (and in general with a self-supervised approach) is that the gauging mechanism is robust enough. Although any neural network (as of now, since there is no clearly established deep learning theory) cannot be guaranteed to perform perfectly well on any data, the top-view person detection is considerably robust. Even if the recall of the top-view person detection is not high, it does not affect the learning approach. It is possible to gauge the precision of the top-view people detected by the top-view person detection and therefore achieve a good working result. It is not necessary to detect all of the people from the top-view.

The output of the depth estimation process is a plan view of the scene and one or more bounding boxes in the plan view around detected people. From the plan view the apparatus computes shortest relative distances 312 between each pair of individuals. In some examples, the apparatus also computes shortest absolute distances 314 between each pair of individuals. More detail about how the shortest relative and absolute distances are computed is given later in this document.

In some examples the estimated shortest distance is a relative distance 312 since it is computed from the plan view alone. In other cases the estimated shortest distance is an absolute distance 314 since additional information is used to enable the estimate to be of absolute distance. The additional information includes one or more attributes of the detected people, where the attributes are computed from the received video frames 300 using computer vision techniques. In an example, an attribute is velocity of a person. Velocity is computed from analysis of change in position of the person in a sequence of frames of the video and using information about a frame rate of the video. In an example, an attribute is change in size of a bounding box of a detected person over frames of the video. Using information about the velocity of a person, together with the change in size of the bounding box and details about parameters of the capture device it is possible to compute an estimate of absolute distance of the person from the capture device.

In a particular example, velocity of a person depicted as walking in the video is assumed to be a specified value such as 1.4 metres per second or other suitable value depending on the application domain. The video is analysed to track a person depicted as walking in the video as early as possible in the video. Two dimensional coordinates in a video-frame depicting the walker are tracked. The video frame rate is known. The difference in the coordinates of the walker between two video frames is calculated and used together with information about the frame rate and the assumed velocity of the walker, to compute an absolute distance corresponding to the difference in the coordinates. A ratio between the coordinates in the video frame and the absolute distance is then known and is used to compute absolute distances between individuals depicted in the plan view.

The estimate of the shortest distance is compared with a threshold. In response to the estimate of the shortest distance being below the threshold a social distancing breach is detected at operation 316. Where the estimate of the shortest distance is a relative value the threshold is scaled according to a homography transformation between the bird's-eye view and the real scene. The threshold is configured by a manufacturer or is configurable by an end user. In an example, a user interface on a touchscreen of a camera is used to configure the threshold. In another example, a cloud or computer based interface is used to configure the threshold.

Where the estimate of the shortest distance is an absolute value the threshold is set according to social distance guidelines of an authority, employer or other entity.

The check at operation 316 is made for each pair of people detected in the video frame. If no social distance breach is found the process returns to operation 300.

In response to detecting a social distancing breach a check is made at operation 318 as to whether to send a push notification. If the video frames received at operation 300 are received from an entity which accepts push notification, such as a smart phone 106, wearable computing device or other mobile computing device, a push notification is triggered 322 and sent to the entity from which the video frames were received. If a rule has been configured indicating that a push notification is to be sent to a particular entity in response to detection of a social distance breach at operation 316 then the rule triggers a notification 322 to be sent to the specified entity. The specified entity is a control center computing device of an authority, employer, site manager or similar.

When a notification is triggered 322 an entry is made in a database 320 which stores information about the detected social distance breach and the associated capture device.

If no push notification is selected at operation 318 the database is updated 320 to indicate the detected social distance breach, the associated capture device and the absence of a push notification.

The method of FIG. 3 includes operations 302 to 308 which occur in parallel with the depth estimation operation 310 and computation of relative or absolute distances 312, 314. Use of parallel processing improves efficiency and enables real time operation.

The video frame is processed by a person detector at operation 302. The person detector is a convolutional neural network having been trained to detect people depicted in video. The person detector is trained using labelled training data and is well known technology. The output of the person detector comprises one or more bounding boxes bounding regions of the video frame depicting a person. A region within a bounding box is processed to compute a facial boundary at operation 304. The region within a bounding box computed as output from operation 302 is input to a neural network face detector which has been trained to compute facial boundaries. The neural network face detector is any convolutional neural network having been trained to detect faces and an example includes the Python Package face recognition (trade mark). The neural network face detector outputs the region within the bounding box in labelled form with image elements labelled as depicting a face or not. In this way a facial boundary is computed 304 as the perimeter of the face region in the output of the neural network face detector.

The facial boundary is optionally expanded at operation 306 to increase the surface area of the region by a specified proportion. This is done, since sometimes the face detection does not cover the full chin or the head area, which may result in subsequent errors in the processing pipeline.

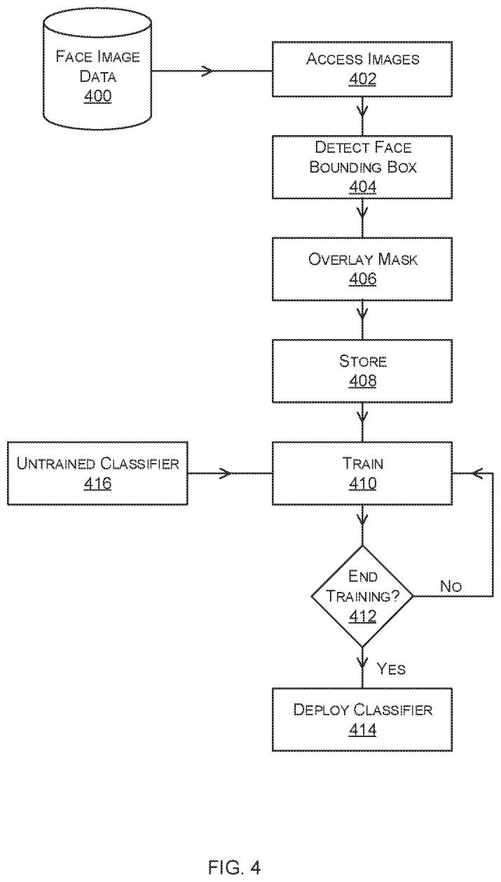

The region within the expanded facial boundary is then analysed to determine whether a face mask or face covering is present or not at operation 308. A trained classifier is used to make the analysis. The classifier is a neural network, support vector machine or other trained machine learning classifier. The classifier has been trained in advance as described in more detail with reference to FIG. 4.

It is found in practice that sometimes the classifier fails due to masks being of varied types, or the faces simply being covered by some valid clothing occlusion such as handkerchiefs/mufflers. In such a case, an additional module is added to detect facial landmarks in each detected face from operation 304. The detection of facial landmarks is done using a deep learning apparatus which outputs the probability of more than 80 landmarks on a facial region. Wherever a face is occluded, the confidence scores of the landmarks there is below a threshold (typically 0.5), and thus the region of occlusion on the face is found. This way, it is possible to detect any protection on the lips and/or the mouth of the person more robustly. However, facial landmark detection is unreliable on very small faces, in which case, the outputs of a classifier are used as described above without a facial landmark detector.

In response to not detecting a face mask or face covering the process moves to operation 318 whereby a decision is made whether or not to make a push notification. In response to detecting a face mask or face covering the process moves to operation 320 whereby the database is updated with information about the detected face mask or face covering and the associated capture device and video frame.

In some examples the method of FIG. 3 is extended to include detection of one or more behaviors indicating that a person is ill, such as sneezing, coughing, shivering, feverish behavior. The behaviors are detected by using a machine learning model which has been trained using videos depicting people exhibiting the behaviors and where the videos are labelled by human judges according to the appropriate behaviors depicted. If one or more of these behaviors are detected the push notification decision 318 is entered and the method proceeds as described above.

In some examples the method of FIG. 3 is extended to include detection of gloves worn by people depicted in the video.

FIG. 4 is a flow diagram of a method of creating training data and a method of using the training data to train a classifier for use in operation 308 of FIG. 3. A database 400 of face image data is accessed where the database has, for many individuals, a plurality of images of the face of the individual with different expressions, poses, lighting conditions, head gear, clothing and other attributes. The face images in database 400 are from a variety of individuals from different demographic groups.

The process accesses 402 an image from the database and inputs the image to a face detector such as that used in operation 304 of FIG. 3. The face detector detects 404 a bounding box indicating a region predicted to depict a face.

Operation 406 comprises overlaying an image of a face mask into the region in the bounding box. In an example, features within the region in the bounding box are detected such as the ears and nose by using line, edge and other feature detectors. A template image of a face mask is then scaled and transformed to fit the detected features and is then overlaid on the region in the bounding box. The shape of the image of the face mask is modified according to the positions of the detected features. A variety of different colours of face mask are generated and used to overlay on the face image data 400.

The resulting modified face image is stored 408.

An untrained classifier 416 is accessed such as a neural network, support vector machine or other classifier. The untrained classifier is trained by supervised training such as backpropagation. The training data comprises the modified face images which are known to depict faces with face masks, face images which are known to depict face coverings which are not masks such as scarves, as well as with the face image data 400 which is known to depict faces without face masks.

A decision is taken at operation 412 whether or not to end training. The criteria for ending training include one or more of: if little or no change in parameter values of the classifier are observed, if a specified number of training iterations have taken place, if the training data is used up.

In response to the training ending the classifier is deployed 414 and available for use in the process of FIG. 3.

FIG. 5 is a flow diagram of a method of operation which is carried out by the apparatus 100 of FIG. 1 and which is more detail of at least part of the depth estimation operation 310 of FIG. 3.

A video frame 500 is accessed and input to a self-supervised machine learning model 502. The video frame 500 depicts a scene with two or more people. In response the trained machine learning model computes a prediction of at least four points in the video frame 500 where the four points depict scene points which are co-planar.

Using the four predicted points the apparatus 100 computes an estimate of a homography 506. A homography is one or more equations or matrices which define a mapping, in this case, from the four predicted points which are co-planar to a plan view of the scene. The homography 506 is not known in advance and so is estimated from the four points using well known geometry calculations. An example of an estimated homography is the following matrix:
[[4.37770414e−01 7.12132115e−01-9.40999930e+021
[−1.54998535e−01 1.04205129e+00 9.64405830e+00]
[−9.11220548e−05 8.12540934e−04 1,00000000e+00]]

Using the homography the video frame 500 is mapped to compute 508 a plan view of the scene. The plan view 510 is input to a person detector 512 which is a neural network that has been trained to detect people in plan view images, such as images captured by drones. In an example, the neural network is implemented using ResNet (trade mark) and trained using the well known VisDrone (trade mark) data set. The output of the person detector 512 is a plurality of bounding boxes 514, one per detected person. Using the bounding boxes 514, shortest distances between pairs of individual bounding boxes are computed 516 to obtain shortest relative distances. The shortest distances are computed as Euclidean distances between the bounding boxes.

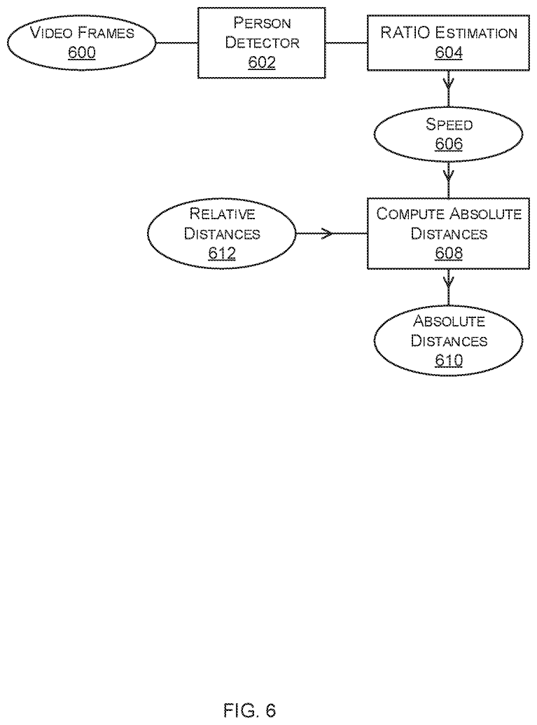

FIG. 6 is a flow diagram of a method of computing shortest absolute distances between a pair of the bounding boxes. Video frames 600 of the video are accessed and a person detector 602 is used to detect one or more people depicted in the video who are walking. The person detector is any neural network having been trained using labelled training data to detect people walking in videos. An estimate of ratio between distance in a video frame and absolute distance in the scene is computed using a ratio estimation process 604. The ratio estimation involves assuming the person is walking at a specified speed. A distance travelled by the person in the video frames is computed during a time interval which is known from the frame rate of the video. The distance travelled during the time interval is compared with the specified speed to determine the ratio.

The ratio is applied to the relative distances 612 from the method of FIG. 5 to convert them to absolute distances 610.

FIG. 7 is a flow diagram of a method of training the self-supervised machine learning model 502 of FIG. 5. The training is of the self-learning type. Unlabeled video training data 700 is available in a database. A frame of training video is accessed 702 and used to compute a forward propagation 704 of a neural network such as a convolutional neural network. The forward propagation produces a plurality of predicted points 706. From a predicted points a homography is estimated 708 and the homography is used to compute a plan view of the scene depicted in the video frame. The plan view is input to a gauging model 712. The gauging model is another neural network which has been trained to detect people in plan views of images.

The output of the gauging model is used to compute a loss 714 and a backpropagation process 716 is used to update the weights of the neural network which is being self-supervised. Thus the loss computed at operation 714 is not a standard loss as is computed in supervised machine learning. In contrast the loss computed at operation 714 is from an output of the gauging model. A check is made at operation 718 to decide whether to end training. Training ends if convergence is reached whereby the update to the weights is small, or if the training data has been used up, or if a specified number of training steps have been completed. If training continues the process returns to operation 702. If training stops the trained model is stored 720.

Using a gauging model to enable self-learning is generally thought to be unreliable. However, it is found that in the present case, since the gauging model is robust its use in the self-learning process of FIG. 7 is practical. In this way it is possible to use self-learning which is a significant benefit because obtaining labelled training data in order to train a neural network for the task of operation 502 is not a practical option.

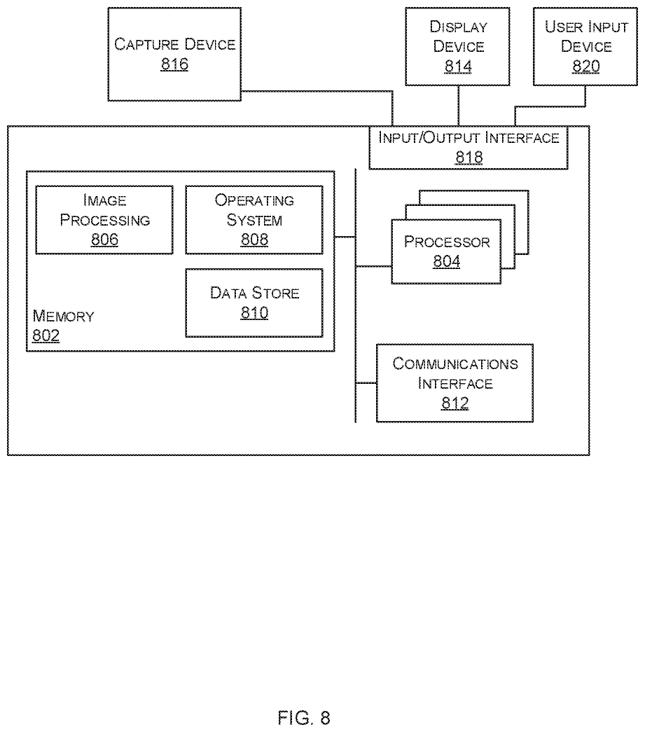

FIG. 8 illustrates various components of an exemplary computing-based device 800 which are implemented as any form of a computing and/or electronic device, and in which embodiments of an image/video analysis apparatus for social distancing applications are implemented in some examples.

Computing-based device 800 comprises one or more processors 804 which are microprocessors, controllers or any other suitable type of processors for processing computer executable instructions to control the operation of the device in order to carry out image processing for social distancing applications 806, detect distance between people depicted in a video, trigger alerts, train a classifier. In some examples, for example where a system on a chip architecture is used, the processors 804 include one or more fixed function blocks (also referred to as accelerators) which implement a part of the method of FIGS. 3 to 7 in hardware (rather than software or firmware). Platform software comprising an operating system 808 or any other suitable platform software is provided at the computing-based device to enable application software to be executed on the device. An image processing application 806 comprises neural network technology and classifier technology to implement the methods of FIGS. 3 to 7. A data store 810 holds image and video data, masks, estimates of absolute and relative distance, thresholds, statistics about social distancing breach events, statistics about face mask absence and other data.

The computer executable instructions are provided using any computer-readable media that is accessible by computing based device 800. Computer-readable media includes, for example, computer storage media such as memory 802 and communications media. Computer storage media, such as memory 802, includes volatile and non-volatile, removable and non-removable media implemented in any method or technology for storage of information such as computer readable instructions, data structures, program modules or the like. Computer storage media includes, but is not limited to, random access memory (RAM), read only memory (ROM), erasable programmable read only memory (EPROM), electronic erasable programmable read only memory (EEPROM), flash memory or other memory technology, compact disc read only memory (CD-ROM), digital versatile disks (DVD) or other optical storage, magnetic cassettes, magnetic tape, magnetic disk storage or other magnetic storage devices, or any other non-transmission medium that is used to store information for access by a computing device, in contrast, communication media embody computer readable instructions, data structures, program modules, or the like in a modulated data signal, such as a carrier wave, or other transport mechanism. As defined herein, computer storage media does not include communication media. Therefore, a computer storage medium should not be interpreted to be a propagating signal per se. Although the computer storage media (memory 802) is shown within the computing-based device 800 it will be appreciated that the storage is, in some examples, distributed or located remotely and accessed via a network or other communication link (e.g. using communication interface 812).

The computing-based device 800 also comprises an input/output interface 818 arranged to output display information to a display device 814 which may be separate from or integral to the computing-based device 800. The display information may provide a graphical user interface such as that indicated in FIG. 1 or the displays of FIG. 2. The input/output interface 818 is also arranged to receive and process input from one or more devices, such as a capture device 816 and one or more user input devices 820. In some examples the user input device 820 detects voice input, user gestures or other user actions.

The term ‘computer’ or ‘computing-based device’ is used herein to refer to any device with processing capability such that it executes instructions. Those skilled in the art will realize that such processing capabilities are incorporated into many different devices and therefore the terms ‘computer’ and ‘computing-based device’ each include personal computers (PCs), servers, mobile telephones (including smart phones), tablet computers, set-top boxes, media players, games consoles, personal digital assistants, wearable computers, and many other devices.

The methods described herein are performed, in some examples, by software in machine readable form on a tangible storage medium e.g. in the form of a computer program comprising computer program code means adapted to perform all the operations of one or more of the methods described herein when the program is run on a computer and where the computer program may be embodied on a computer readable medium. The software is suitable for execution on a parallel processor or a serial processor such that the method operations may be carried out in any suitable order, or simultaneously.

This acknowledges that software is a valuable, separately tradable commodity. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

Those skilled in the art will realize that storage devices utilized to store program instructions are optionally distributed across a network. For example, a remote computer is able to store an example of the process described as software. A local or terminal computer is able to access the remote computer and download a part or all of the software to run the program. Alternatively, the local computer may download pieces of the software as needed, or execute some software instructions at the local terminal and some at the remote computer (or computer network). Those skilled in the art will also realize that by utilizing conventional techniques known to those skilled in the art that all, or a portion of the software instructions may be carried out by a dedicated circuit, such as a digital signal processor (DSP), programmable logic array, or the like.

Any range or device value given herein may be extended or altered without losing the effect sought, as will be apparent to the skilled person.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items.

The operations of the methods described herein may be carried out in any suitable order, or simultaneously where appropriate. Additionally, individual blocks may be deleted from any of the methods without departing from the scope of the subject matter described herein. Aspects of any of the examples described above may be combined with aspects of any of the other examples described to form further examples without losing the effect sought.

The term ‘comprising’ is used herein to mean including the method blocks or elements identified, but that such blocks or elements do not comprise an exclusive list and a method or apparatus may contain additional blocks or elements.

It will be understood that the above description is given by way of example only and that various modifications may be made by those skilled in the art. The above specification, examples and data provide a complete description of the structure and use of exemplary embodiments. Although various embodiments have been described above with a certain degree of particularity, or with reference to one or more individual embodiments, those skilled in the art could make numerous alterations to the disclosed embodiments without departing from the scope of this specification.

What is claimed is:

1. A computer-implemented method comprising:
storing in a memory, a video captured by a capture device, the video depicting a scene comprising two or more people in an environment;
inputting at least one frame of the video to a self-supervised neural network and in response, receiving a prediction of at least four points in the frame which depict four points on a plane of the scene;
computing, from the four points, a plan view of the scene;
detecting two or more people in the plan view of the scene; and
computing, for individual pairs of people depicted in the plan view, an estimate of shortest distance between the people in the pair.

2. The method of claim 1 comprising, in response to at least one of the estimated shortest distances being below a threshold, triggering an alert.

3. The method of claim 2 wherein the alert is a message sent to a control apparatus associated with the capture device and/or a push notification sent to a mobile computing device.

4. The method of claim 1 wherein the estimate of shortest distance is a relative distance.

5. The method of claim 1 wherein the estimate of shortest distance is an absolute distance computed based on attributes of the people computed from the video, the attributes comprising velocity and/or change in size of a bounding box of each person depicted in the video.

6. The method of claim 1 wherein the estimate of shortest distance is an absolute distance computed based on a specified walking speed and a frame rate of the video.

7. The method of claim 1 wherein the neural network has been trained without labeled training data.

8. The method of claim 1 wherein the neural network has been trained using a gauge model comprising a neural network trained to detect people from images taken by drones.

9. The method of claim 1 wherein computing, from the four points, a plan view of the scene comprises using estimating a homography and using the homography to transform the video frame into the plan view of the scene.

10. The method of claim 1 comprising:
inputting the at least one frame of the video to a neural network trained to detect faces, and receiving as output a facial detection boundary for each face detected in the frame, a facial detection boundary comprising a face region;
for each face region, inputting the face region to a classifier trained to detect face masks to compute a classification of the face region as depicting a face mask or not.

11. The method of claim 10 comprising expanding each face region before inputting the expanded face region to the classifier.

12. The method of claim 10 comprising triggering the alert in response to at least one of the face regions being classified as not depicting a face mask in addition to one of the estimated shortest distances being below a threshold triggering an alert.

13. The method of claim 10 comprising triggering a push notification in response to at least one of the face regions being classified as not depicting a face mask.

14. The method of claim 10 comprising training the classifier using training data comprising real images of faces which have been modified by overlay of a face mask as well as real images of faces with no face mask.

15. The method of claim 14 wherein overlay of a face mask comprises identifying features of the face and modifying the shape of the image of the face mask according to the positions of the identified features.

16. The method of claim 14 comprising modifying the real images of faces by overlaying images of a face mask using a variety of different colours of images of face mask.

17. An apparatus comprising:
a memory storing a video captured by a capture device, the video depicting a scene comprising two or more people in an environment;
a self-supervised neural network which takes at least one frame of the video as input and in response, computes a prediction of at least four points in the frame which depict four points on a plane of the scene;
a processor which computes, from the four points a homography which is used to compute a plan view of the scene, and the processor detects two or more people in the plan view of the scene; and
where the processor computes, for individual pairs of people depicted in the plan view, an estimate of shortest distance between the people in the pair.

18. The apparatus of claim 17 wherein the capture device is integral with the apparatus.

19. The apparatus of claim 17 wherein the estimate of shortest distance is computed as a Euclidean distance in the plan view.

20. One or more device-readable media with device-executable instructions that, when executed by a computing system, direct the computing system to perform for performing operations comprising:
storing in a memory, a video captured by a capture device, the video depicting a scene comprising two or more people in an environment;
inputting at least one frame of the video to a self-supervised neural network and in response, receiving a prediction of at least four points in the frame which depict four points on a plane of the scene;
computing, from the four points, a plan view of the scene;
detecting two or more people in the plan view of the scene;
computing, for individual pairs of people depicted in the plan view, an estimate of shortest distance between the people in the pair; and
in response to the estimate of shortest distance being below a threshold, triggering an alert.
